(19) United States
(12) Patent Application Publication
(10) Pub. No.: US 2008/0243175 A1
(43) Pub. Date: Oct. 2, 2008

(54) Surgical Gripping Tool Having Dual-Linkage, Force Multiplying Coupler and Shaped to Grip Multiple Size Rods

(75) Inventors: Jesse Gabriel Moore, Swartz Creek, MI (US); Rodney Ray Ballard, Lakeland, TN (US); William Alan Rezach, Atoka, TN (US)

Correspondence Address: COATS & BENNETT/MEDTRONIC, 1400 CRESCENT GREEN, SUITE 300, CARY, NC, 27518, US

(73) Assignee: Warsaw Orthopedic, Inc., Warsaw, IN (US)

(21) Appl. No.: 11/692,516

(22) Filed: Mar. 28, 2007

(51) Int. Cl. A61B 17/00; A61B 17/58

(52) U.S. Cl. 606/206; 606/205

(57) ABSTRACT

A surgical gripping tool includes a dual-linkage, force multiplying coupling that increases the force applied to surgical rods for a given force applied to the tool handles. In some embodiments, recesses in the tool that grip surgical rods have complex surfaces comprising multiple radii, allowing the tool to securely grip surgical rods of different diameters. The recess radii may be undersized relative to the associated rod size. In one embodiment, the coupling multiplies an applied force by a factor of over 20.

### BACKGROUND

The present application relates generally to surgical hand tools and in particular to a surgical gripping tool having a dual-linkage, force multiplying coupler and shaped to grip multiple size rods.

A known type of spinal osteosynthesis involves securing fasteners—such as sacral screws, pedicle screws, transverse connectors, bone hooks, and the like—to the spine, and connecting one or more pre-shaped surgical rods to the fasteners, conforming or urging the spine to the shape of the rods. In a common procedure, the fasteners have a spinal rod receiving bore extending through a head or other protruding portion. The fasteners are secured to the spine at desired locations, and a spinal rod in a desired shape is then extended through the spinal rod bore in each fastener. Set screws in the fasteners may then be tightened to prevent translational and/or rotational movement of the rods within the bores. The rods exert the desired force on the spine, urging it to the shape of the rods. Considerable force must be applied to the rods to align and install rods through the fastener bores. Conventional surgical gripping tools require a powerful grip by the surgeon to exert sufficient force on surgical rods to hold the rods during a spinal osteosynthesis procedure. Additionally, conventional surgical gripping tools are “sized” for specific diameter surgical rods, requiring a separate surgical gripping tool for each size of rod used in a spinal osteosynthesis procedure.

### SUMMARY

According to one or more embodiments, a surgical gripping tool includes a dual-linkage, force multiplying coupling that increases the force applied to surgical rod. In one embodiment, the coupling multiplies an applied force by a factor of over 20. In some embodiments, recesses that grip surgical rods have complex surfaces comprising multiple radii, allowing the tool to securely grip surgical rods of different diameters. The recess radii may be undersized relative to the associated rod size.

In one embodiment, the present application relates to a surgical gripping tool. The tool includes two generally elongate handle members, each having a handle end and a pivot end. The handle members are pivotally connected to each other at a first pivot point closer to the pivot end than to the handle end. The tool further includes two generally elongate gripping members, each having a pivot end and a gripping end. The gripping members are pivotally connected to each other at a second pivot point closer to the gripping end than to the pivot end. Each gripping member is pivotally connected to a handle member at their respective pivot ends.

In another embodiment, the present application relates to a method of performing spinal osteosynthesis surgery. A plurality of fasteners are attached to a spine, each fastener including a spinal rod receiving bore. A spinal rod is gripped with a surgical gripping tool having a dual-linkage coupling. The spinal rod is inserted through the spinal rod receiving bores of two or more fasteners.

In yet another embodiment, the present application relates to a surgical gripping tool. The tool includes two handles and jaws adapted to grip and hold surgical rods. The tool also includes a dual-linkage, force-multiplying coupling mechanism connecting the handles to the jaws and operative to apply to the jaws a multiple of the force applied to the handles.

### BRIEF DESCRIPTION OF THE DRAWINGS

FIG. 1 is a perspective view of a surgical gripping tool according to one embodiment.

FIG. 2 is a section view of a rod-gripping recess in the surgical gripping tool of FIG. 1.

### DETAILED DESCRIPTION

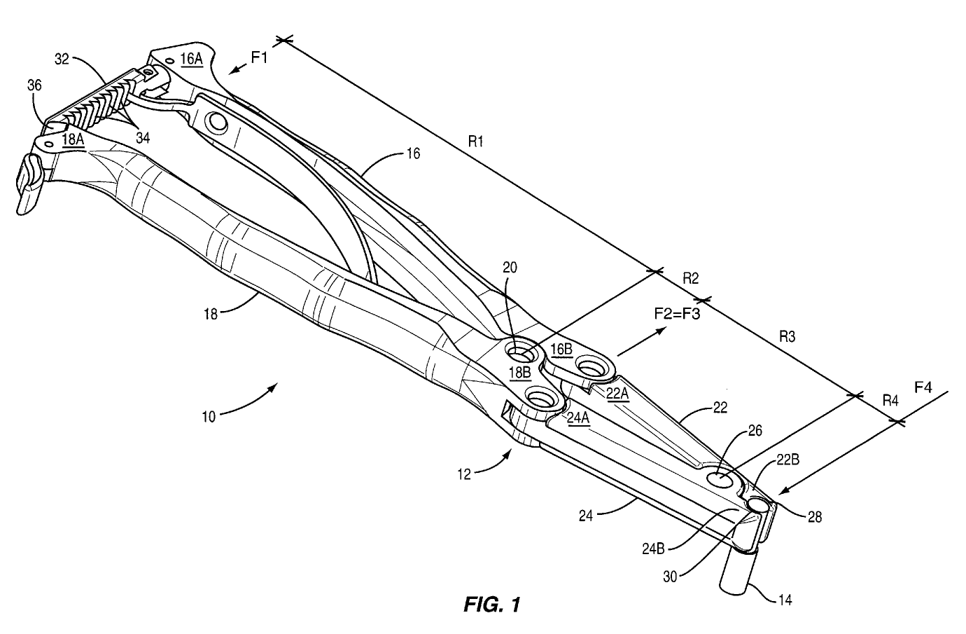

FIG. 1 depicts a surgical rod-gripping hand tool, indicated generally at 10, according to one embodiment. The tool 10 includes a dual-linkage, force-multiplying coupling 12. The coupling 12 applies much greater force to a rod 14 in its grip, than the force applied to the tool 10 by a surgeon.

The tool 10 includes two generally elongate handle members 16, 18, each having a handle end A and a pivot end B. The handle members 16, 18 are connected together at a pivot point 20, and pivot about the pivot point 20. Note that the handle members 16, 18 do not cross at the pivot point 20. Accordingly, as the handle ends 16A, 18A of the handle members 16, 18 move towards each other, the pivot ends 16B, 18B move apart from each other. As discussed in greater detail herein, the pivot point 20 is closer to the pivot ends 16B, 18B of the handle members 16, 18 than it is to the handle ends 16A, 18A.

The tool 10 further includes two generally elongate gripping members 22, 24, each having a pivot end A and a gripping end B. The gripping members 22, 24 are each pivotally connected to the handle members 16, 18 at their respective pivot ends 22A, 24A, 16B, 18B. In particular, the pivot end 22A of the gripping member 22 is pivotally connected to the pivot end 16B of the handle member 16. Similarly, the pivot end 24A of the gripping member 24 is pivotally connected to the pivot end 18B of the handle member 18. The gripping members 22, 24 are connected together at a pivot point 26, and pivot about the pivot point 26. Like the handle members 16, 18, the gripping members 22, 24 do not cross at the pivot point 20. Accordingly, as the pivot ends 22A, 24A of the gripper members move away from each other, the gripping ends 22B, 24B move toward each other. As discussed in greater detail herein, the pivot point 26 is closer to the gripping ends 22B, 24B of the gripping members 22, 24 than it is to the pivot ends 22A, 24A.

In use, the handle ends 16A, 18A of the handle members 16, 18 are moved apart from each other to open the tool 10. In so doing, the handle members 16, 18 pivot about the pivot point 20, moving the pivot ends 16B, 18B of the handle members 16, 18 towards each other. This also moves the pivot ends 22A, 24A of the gripping members 22, 24 towards each other. The gripping members 22, 24 pivot about the pivot point 26, moving the gripping ends 22B, 24B of the gripping members 22, 24 away from each other. This opens the tool to grip a surgical rod 14.

Formed in the gripping ends 22B, 24B of the gripping members 22, 24 are generally semi-circular recesses 28, 30, respectively. As discussed in greater detail herein, in one or more embodiments each recesses 28, 30 may have a complex surface with different radii, to provide a firm grip on surgical rods having different diameters. A surgical rod is placed within the “jaws” of the tool 10 formed by the recesses 28, 30 in the gripping ends 22B, 24B of the gripping members 22, 24, or alternatively the jaws of the tool 10 are placed around a surgical rod already installed in a patient. In one embodiment, the semi-circular recesses 28, 30 have radii slightly undersized relative to the corresponding rod diameters, to provide and improved grip.

Closing the tool 10 to grip a rod 14 is a straightforward reversal of the procedure to open the tool 10. In particular, the handle ends 16A, 18A of the handle members 16, 18 are moved towards each other. In so doing, the handle members 16, 18 pivot about the pivot point 20, moving the pivot ends 16B, 18B of the handle members 16, 18 apart from each other. This also moves the pivot ends 22A, 24A of the gripping members 22, 24 apart from each other. The gripping members 22, 24 pivot about the pivot point 26, moving the gripping ends 22B, 24B of the gripping members 22, 24 towards each other and closing on the rod 14. In the same manner, by applying a force, depicted in FIG. 1 as F₁, urging the handle ends 16A, 18A of the handle members 16, 18 together, the tool 10 applies a dramatically multiplied force F₄ to grip the rod 14 in the rod holding recesses 28, 30.

The coupling 12 of the surgical gripping tool 10 is thus a “dual-linkage” type, with handle members 16, 18 linked together at pivot point 20, and gripping members 22, 24 linked together at pivot point 26. Intermediate the pivot points 20, 26, the handle members 16, 18 are pivotally connected to the gripping members 22, 24, respectively. The dual-linkage coupling 12 multiplies the force exerted on the handle members 16, 18 to a greater extent than prior art, single-linkage coupling designs, in applying the force to a surgical rod 14 within the rod holding recesses 28, 30 of the gripping ends 22B, 24B of the tool 10.

In one embodiment, a locking arm 32 having at least one notch 34 (and preferably a plurality of notches 34) is attached to the handle end 16A of one handle member 16. A locking tab 36 is attached to the handle end 18A of the other handle member 18. In another embodiment, the locking arm 32 may be attached to the handle member 18 and the locking tab 36 may be attached to the handle member 16. With the tool 10 in the closed, gripping position, the locking tab 36 may engage a notch 34 in the locking arm 32. This holds the handle ends 16A, 18A together, maintaining a force F₄ on a rod 14 without a surgeon constantly applying a force F₁ to the handle ends 16A, 18A of the handle members 16, 18.

To demonstrate the force multiplying feature of the coupling 12 of the tool 10, FIG. 1 depicts force vectors and distances (radii from the pivot points 20, 26) for a Free Body Diagram analysis of the tool 10. Surgical rods are generally formed of steel, titanium, or other metal, and are not appreciably deformable in the radial direction of a cross-section. Accordingly, once the tool 10 is in the closed position and gripping a rod 14, the members 16, 18, 22, 24 do not move with respect to each other as the applied force F₁ increases. Static equilibrium requires that the sum of moments about the pivot points 20, 26 is zero. In particular, the torque or moment F₁R₁ about pivot point 20 in a counterclockwise direction (as depicted in FIG. 1) must be exactly balanced by the moment F₂R₂ in a clockwise direction, or

F₁R₁=F₂R₂ (1)

Similarly, the moments about pivot point 26 must sum to zero. In particular, the torque or moment F₃R₃ about pivot point 26 in a clockwise direction must be exactly balanced by the moment F₄R₄ in a counterclockwise direction, or

F₃R₃=F₄R₄ (2)

Since the pivot ends 22A, 24A of the gripping the members 22, 24 are mechanically coupled to the pivot ends 16B, 18B of the handle members 16, 18, they necessarily experience the same force. Thus,

F₂=F₃ (3)

Solving eq. (1) for F₂,

$${F}_{2} = \frac{{F}_{1} {R}_{1}}{{R}_{2}}$$

Using the identity of equation (3), and substituting for F₃ in equation (2) yields

$$( \frac{{F}_{1} {R}_{1}}{{R}_{2}} ) {R}_{3} = {F}_{4} {R}_{4} or \\ {F}_{4} = {F}_{1} ( \frac{{R}_{1} {R}_{3}}{{R}_{2} {R}_{4}} ) \qquad ( 4 )$$

Equation (4) expresses the gripping force F₄ applied to the rod 14 as a multiple of the force F₁ applied to the tool 10 by a surgeon. If the pivot point 20 of the handle members 16, 18 is closer to the pivot end 16B, 18B than to the handle end 16A, 18A, R₁>R₂. Similarly, if the pivot point 26 of the gripping members 22, 24 is closer to the gripping end 22B, 24B than to the pivot end 22A, 24A, R₃>R₄. In the embodiment depicted in FIG. 1, R₁>>R₂ and R₃>>R₄. Accordingly, R₁R₃>>R₂R₄, and the force multiplying factor of equation (4) is large.

In one embodiment, the values of R₁-R₄ are [in mm]: R₁=110, R₂=20, R₃=35, and R₄=20. The total length of the tool 10 in this embodiment is 185 mm. If a force of 100 N is applied as F₁, the force F₂=F₃ is 550 N and the gripping force F₄ is 962.5 N. This yields a force ratio or multiple of 9.6 (i.e., F₄=9.6 F₁).

In another embodiment, R₁=150, R₂=30, R₃=45, and R₄=10 for a total length of 235 mm. If a force of 100 N is applied as F₁, the force F₂=F₃ is 500 N and the gripping force F₄ is 2250 N. This yields a force multiple of 22.5 (i.e., F₄=22.5 F₁). Given the teachings herein, those of skill in the art may readily alter the relative lengths of handle members 16, 18 and gripping members 22, 24, and the placement of pivot points 20, 26, to achieve a desired force multiple within the size and weight constraints of a particular application. As further non-limiting examples, Table 1 lists a plurality of dimensions for a surgical gripping tool of the type depicted in FIG. 1.

TABLE 1
Representative Dimensions of Surgical Gripping Tool

| | Total | | | | | | Force |
|---|---|---|---|---|---|---|---|
| F₁ | Length | R₁ | R₂ | R₃ | R₄ | F₄ | Multiple |
| 100 | 222 | 145 | 33 | 35 | 9 | 1709 | 17.1 |
| 100 | 222 | 145 | 32 | 36 | 9 | 1813 | 18.1 |
| 100 | 222 | 145 | 31 | 37 | 9 | 1923 | 19.2 |
| 100 | 222 | 145 | 30 | 38 | 9 | 2041 | 20.4 |
| 100 | 222 | 145 | 29 | 39 | 9 | 2167 | 21.7 |
| 100 | 222 | 145 | 28 | 40 | 9 | 2302 | 23.0 |
| 100 | 222 | 145 | 27 | 41 | 9 | 2447 | 24.5 |
| 100 | 222 | 145 | 26 | 42 | 9 | 2603 | 26.0 |
| 100 | 222 | 145 | 25 | 43 | 9 | 2771 | 27.7 |

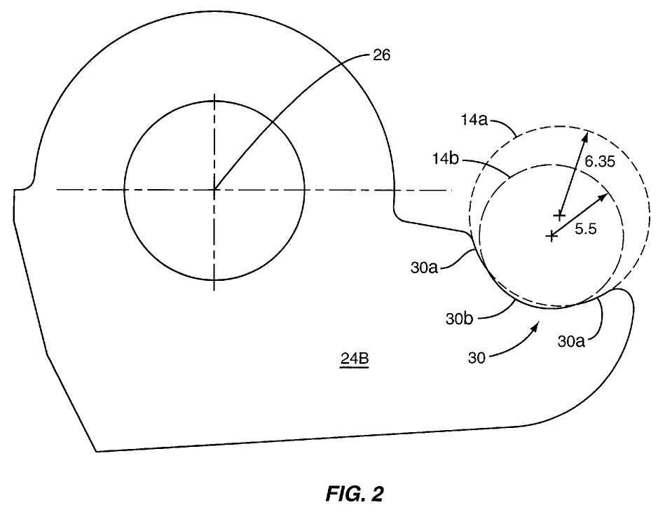

In one or more embodiments, the recesses 28, 30 formed in the gripping ends 22B, 24B of griping members 22, 24 have a complex semi-circular surface comprising at least two different radii, to provide a firm grip on surgical rods having different diameters. This is depicted in FIG. 2, showing a section view of the gripping end 24B of gripping member 24. The recess 30 has first surfaces 30a sized to fit a first surgical rod 14a having a diameter of 6.35 mm. In one embodiment, the diameter of surface 30a is slightly undersized, having a diameter of 6.25 mm. The rod 14a contacts the surfaces 30a on either side of a second surface 30b. The second surface 30b is sized to fit a second surgical rod 14b having a diameter of 5.5 mm. In one embodiment, the diameter of surface 30b is slightly undersized, having a diameter of 5.4 mm. The rod 14b fits within the secondary recess of surface 30b, and does not contact all of surfaces 30a. In this manner, a single, complex recess surface 30 may contact and firmly grip different sized rods 14a and 14b, as well as rods sized intermediate to 14a and 14b.

The present application may, of course, be carried out in other ways than those specifically set forth herein without departing from essential characteristics. The present embodiments are to be considered in all respects as illustrative and not restrictive, and all changes coming within the meaning and equivalency range of the appended claims are intended to be embraced therein.

What is claimed is:

1. A surgical gripping tool, comprising:
two generally elongate handle members, each having a handle end and a pivot end, the handle members pivotally connected to each other at a first pivot point closer to the pivot end than the handle end; and
two generally elongate gripping members, each having a pivot end and a gripping end, the gripping members pivotally connected to each other at a second pivot point closer to the gripping end than the pivot end, each gripping member pivotally connected to a handle member at their respective pivot ends.

2. The tool of claim 1 wherein both the handle members and gripping members are respectively connected to each other without crossing, whereby the ends of the members on opposite sides of the pivot points move in opposite directions when pivoted about the pivot points.

3. The tool of claim 1 wherein moving the handle ends of the handle members together moves the pivot end of the handle members apart and also moves the pivot ends of the gripping members apart, moving the gripping ends of the gripping members together.

4. The tool of claim 3 wherein the tool exhibits a force multiplier whereby the gripping ends of the gripping members move together with a greater force than the force exerted to move the handle ends of the handle members together.

5. The tool of claim 4 where in the force multiplier is in the range from about 17 to about 28.

6. The tool of claim 4 where in the force multiplier is in the range from about 20 to about 26.

7. The tool of claim 4 where in the force multiplier is about 22.5.

8. The tool of claim 1 wherein a generally semi-circular recess is formed in the gripping end of each gripping member to facilitate holding a rod.

9. The tool of claim 8 wherein each recess has a complex surface having different radii to facilitate holding rods of different diameter.

10. The tool of claim 9 wherein each recess is shaped to hold surgical rods of at least 5.5, 6.0, and 6.35 mm, +/−0.5 mm.

11. The tool of claim 9 wherein the complex surface radii are undersized relative to the associated rod diameter.

12. The tool of claim 1 further comprising a locking arm having at least one notch attached to the handle end of one handle member and a locking tab attached to the handle end of the other handle member, the tab operative to engage a notch in the locking arm in at least one position along the locking arm to lock the tool in a closed position.

13. The tool of claim 11 wherein the locking arm has a plurality of notches corresponding to a plurality of positions of the handle members, each position generating a different force at the griping ends of the gripping members and wherein the tab is operative to selectively engage one of the plurality of notches to selectively maintain a corresponding amount of force.

14. A method of performing spinal osteosynthesis surgery, comprising:
attaching a plurality of fasteners to a spine, each fastener including a spinal rod receiving bore;
gripping a spinal rod with a surgical gripping tool having a dual-linkage coupling; and
inserting the spinal rod through the spinal rod receiving bores of two or more fasteners.

15. The method of claim 14 wherein gripping a spinal rod with a surgical gripping tool having a dual-linkage coupling comprises gripping the rod within rod-receiving recesses formed in the tool, each recess comprising a generally semi-circular, complex surface having different radii to facilitate holding rods of different diameter.

16. The method of claim 15 wherein the rod-receiving recesses are shaped to hold surgical rods of at least 5.5, 6.0, and 6.35 mm, +/−0.5 mm.

17. The method of claim 15 wherein the complex surface radii are undersized relative to the associated rod diameter.

18. A surgical gripping tool, comprising:
two handles;
jaws adapted to grip and hold surgical rods; and
a dual-linkage, force-multiplying coupling mechanism connecting the handles to the jaws and operative to apply to the jaws a multiple of the force applied to the handles.

19. The tool of claim 18 wherein each jaw includes generally semi-circular, complex surface recesses formed therein and adapted to grip and hold surgical rods of at least 5.5, 6.0, and 6.35 mm, +/−0.5 mm.

20. The tool of claim 18 wherein the complex surface radii are undersized relative to the associated rod diameter.
